(19) United States
(12) Patent Application Publication
(10) Pub. No.: US 2021/0085695 A1
(43) Pub. Date: Mar. 25, 2021

(54) METHODS AND COMPOSITIONS FOR THE TREATMENT OF MIGRAINE HEADACHES

(71) Applicant: Beth Israel Deaconess Medical Center, Inc., Boston, MA (US)

(72) Inventors: Rami BURSTEIN, Chesnut Hill, MA (US); Catherine BUETTNER, Boston, MA (US)

(21) Appl. No.: 17/061,171

(22) Filed: Oct. 1, 2020

Related U.S. Application Data
(63) Continuation of application No. 14/282,132, filed on May 20, 2014, now abandoned.
Application No. 14/282,132 is a division of application No. 13/520,054, filed on Sep. 17, 2012, now abandoned.
(60) Provisional application No. 61/393,680, filed on Oct. 15, 2010.
(60) Provisional application No. 61/293,379, filed on Jan. 8, 2010.

(51) Int. Cl. A61K 31/593; A61K 31/59; A61K 45/06; A61K 31/366; A61K 31/351; A61K 31/592

(52) U.S. Cl. CPC A61K 31/593; A61K 31/59; A61K 31/592; A61K 31/366; A61K 31/351; A61K 45/06

(57) ABSTRACT

The present invention is related to compositions and methods for treating or reducing the likelihood of a migraine, reducing the severity of migraine, reducing the frequency of migraines, reducing the duration of migraine, and ameliorating the symptoms of a migraine. The methods and compositions of the present invention may also be used to treat or prevent condition characterized by increased cardiovascular risk or endothelial dysfunction and musculoskeletal symptoms.

### BACKGROUND OF THE INVENTION

Migraine headache is associated with dural and meningeal vascular dilatation and perivascular inflammation. The vasculopathy of migraines is thought to reflect endothelial dysfunction, a disorder of endothelial activation and impaired vascular reactivity. Endothelial activation gives rise to inflammation and thrombosis, while impaired vascular reactivity reduces bioavailability of vasodilators and increases the level of endothelium-derived contracting factors. Migraine sufferers may also experience abnormal muscle tenderness during migraine episodes. Muscle tenderness may be chronic, may appear several hours before the onset of a migraine attack, or may appear several hours after the onset of migraine.

Current migraine therapies are inadequate for treating migraine sufferers who are intolerant to such therapies or for those who suffer from frequent, severe, or intractable migraine pain. While several classes of prophylactic medications are available to treat this population, many of the existing migraine therapies are minimally effective or are not well tolerated. In addition, many individuals who experience migraine are at increased risk for cardiovascular events, which are not addressed by current prophylactics.

Thus, there exists a need in the art for therapies that treat, ameliorate, and/or prevent migraines, associated symptoms, and cardiovascular risk, such as stroke.

### SUMMARY OF THE INVENTION

The present invention is related to compositions and methods for treating or reducing the likelihood of a migraine, reducing the frequency of migraines, ameliorating the symptoms of a migraine, and reducing cardiovascular risk. The methods and compositions of the present invention may also be used to treat or prevent conditions characterized by increased cardiovascular risk or endothelial dysfunction and musculoskeletal symptoms.

In a first aspect, the invention features a method of treating or reducing the likelihood of a migraine in a subject (e.g., a human) in need thereof. The method includes administering to a subject (i) a statin or analogs thereof and (ii) vitamin D or analogs thereof, wherein a statin or analogs thereof and vitamin D or analogs thereof are administered in amounts and for a duration that together are sufficient to treat or reduce the likelihood of a migraine in a subject.

In a second aspect, the invention features a method of ameliorating the symptoms of a migraine in a subject (e.g., a human) in need thereof. The method includes administering to a subject (i) a statin or analogs thereof and (ii) vitamin D or analogs thereof, wherein a statin or analogs thereof and vitamin D or analogs thereof are administered in amounts and for a duration that together are sufficient to ameliorate the symptoms of a migraine in a subject.

In a third aspect, the invention features a method of reducing the frequency or duration of migraines in a subject (e.g., a human) in need thereof. The method includes administering to a subject (i) a statin or analogs thereof and (ii) vitamin D or analogs thereof, wherein a statin or analogs thereof and vitamin D or analogs thereof are administered in amounts and for a duration that together are sufficient to reduce the frequency or duration of migraines in a subject.

In a fourth aspect, the invention features a method of treating a condition characterized by endothelial dysfunction and musculoskeletal symptoms in a subject (e.g., a human) in need thereof. The method includes administering to a subject (i) a statin or analogs thereof and (ii) vitamin D or analogs thereof, wherein a statin or analogs thereof and vitamin D or analogs thereof are administered in amounts and for a duration that together are sufficient to treat a condition characterized by endothelial dysfunction and musculoskeletal symptoms. In one embodiment, the condition is statin-associated musculoskeletal pain or weakness, arthritis, neuromuscular disease, a condition associated with stroke rehabilitation, or a condition associated with myocardial infarction rehabilitation.

In one embodiment of the first, second, third, or fourth aspect, the statin is atorvastatin; cerivastatin; fluvastatin; lovastatin; rosuvastatin; acitemate; amlodipine/atorvastatin; BAY102987; BAY X 2678; BB476; bervastatin; BMY21950; BMY22089; colestolone; CP83101; dalvastatin; DMP565; ezetimibe/simvastatin; glenvastatin; L659699; L669262; mevastatin; nicotinic acid/lovastatin; nicotinic acid/simvastatin; P882222; P882284; PD134965; PD135022; pitavastatin; rosuvastatin; RP61969; S2468; SC37111; SC45355; simvastatin; SQ33600; SR12813; SR45023A; U20685; and U88156. In another embodiment, vitamin D or the vitamin D analog is doxercalciferol, calcitriol, α-calcidol, seocalcitol, calcipotriol, maxacalcitol, falecalcitriol, paricalcitol, alfacalcidol, calcifediol, cholecalciferol, dihydrotachysterol, ergosterol, ergocalciferol, 1α,2-dihydroxyvitamin D₄, 1α,24-dihydroxyvitamin D₂, 1α,25-dihydroxyvitamin D₂, 1α,25-dihydroxyvitamin D₄, or 1α,24,25-dihydroxyvitamin D₂.

In another embodiment of the first, second, third, or fourth aspect, the statin is simvastatin or the vitamin D or vitamin D analog is ergocalciferol and/or cholecalciferol. In a particular embodiment, the statin is simvastatin and the vitamin D or vitamin D analog is ergocalciferol or cholecalciferol.

In one embodiment of any of the above aspects, a statin or analogs thereof and vitamin D or analogs thereof are administered simultaneously or sequentially.

In a fifth aspect, the invention features a pharmaceutical composition that includes (i) a statin or analogs thereof, (ii) vitamin D or analogs thereof, and (iii) a pharmaceutically acceptable carrier, wherein a statin or analogs thereof and vitamin D or analogs thereof are present in an amount that, when administered to a subject, are sufficient to treat or reduce the likelihood of a migraine in a subject.

In a sixth aspect, the invention features a pharmaceutical composition that includes (i) a statin or analogs thereof, (ii) vitamin D or analogs thereof, and (iii) a pharmaceutically acceptable carrier, wherein a statin or analogs thereof and vitamin D or analogs thereof are present in an amount that, when administered to a subject, are sufficient to ameliorate the symptoms of a migraine in a subject.

In a seventh aspect, the invention features a pharmaceutical composition that includes (i) a statin or analogs thereof; (ii) vitamin D or analogs thereof, and (iii) a pharmaceutically acceptable carrier, wherein a statin or analogs thereof and vitamin D or analogs thereof are present in an amount that, when administered to a subject, are sufficient to reduce the frequency or duration of migraines in a subject.

In an eighth aspect, the invention features a pharmaceutical composition that includes (i) a statin or analogs thereof; (ii) vitamin D or analogs thereof, and (iii) a pharmaceutically acceptable carrier, wherein a statin or analogs thereof and vitamin D or analogs thereof are present in an amount that, when administered to a subject, are sufficient to treat a condition characterized by increased cardiovascular risk or endothelial dysfunction and musculoskeletal symptoms in a subject. In one embodiment, the condition is statin-associated musculoskeletal pain or weakness, arthritis, neuromuscular disease, a condition associated with stroke rehabilitation, or a condition associated with myocardial infarction rehabilitation.

In one embodiment of the fifth, sixth, seventh, or eighth aspect, a statin or analogs thereof and vitamin D or analogs thereof are formulated in a single composition. The pharmaceutical compositions may be formulated for oral administration or systemic administration. Such pharmaceutical compositions may be formulated for inhalation or for topical administration.

In another embodiment of the fifth, sixth, seventh, or eighth aspect, the statin is simvastatin or the vitamin D or vitamin D analog is ergocalciferol and/or cholecalciferol. In a particular embodiment, the statin is simvastatin and the vitamin D or vitamin D analog is ergocalciferol or cholecalciferol.

In one embodiment of any of the above aspects, an additional therapeutic agent is administered to a subject or present in a pharmaceutical composition. The additional therapeutic agent may be an analgesic (e.g., a non-steroidal anti-inflammatory agent), a β-blocker, a calcium channel blocker, an antiemetic, a serotonin receptor agonist, a barbiturate, an antidepressant (e.g., a tricyclic antidepressant), an ergot alkaloid, a steroid, an anxiolytic, an amphetamine, a NOS inhibitor, a narcotic, or an anticonvulsant (e.g., valproic acid).

By “an amount sufficient” is meant the amount of a compound or therapeutic agent, alone or in combination with another compound or therapeutic regimen, required to treat or ameliorate a disorder, such as a migraine headache, in a clinically relevant manner. A sufficient amount of an active compound or therapeutic agent used to practice the present invention for therapeutic treatment of, e.g., a migraine headache varies depending upon the manner of administration, age, and general health of the subject. Ultimately, the medical practitioner prescribing such treatment will decide the appropriate amount and dosage regimen. Additionally, an effective amount may be an amount of compound in the combination of the invention that is safe and efficacious in the treatment of a subject having a disorder, such as a migraine headache, over each agent alone as determined and approved.

By “analog” is meant is a molecule that differs from, but is structurally, functionally, and/or chemically related to the reference molecule (i.e., a statin or vitamin D). The analog may retain the essential properties, functions, or structures of the reference molecule. Most preferably, the analog retains at least one biological function of the reference molecule. Generally, differences are limited so that the structure or sequence of the reference molecule and the analog are similar overall. An analog of a statin or vitamin D may be naturally occurring, or it may be a variant that is not known to occur naturally. Non-naturally occurring analogs of a statin or vitamin D may be made synthetically or by modification.

By “cardiovascular risk” is meant factors that research has shown increase the risk of heart and blood vessel (i.e., cardiovascular) disease, such as hypertension, high cholesterol, diabetes, metabolic syndrome, increased C-reactive protein, or other signs of systemic inflammation.

By “endothelial dysfunction” is meant a physiological dysfunction of normal biochemical processes carried out by the endothelium. Normal functions of endothelial cells include, for example, mediation of coagulation, platelet adhesion, and immune function.

By “migraine” is meant a subset of headaches characterized by unusually severe, unilateral, throbbing head pain that often includes additional symptoms described herein. Migraine is meant to include, for example, migraine without aura (e.g., common migraine), migraine with aura (e.g., classical migraine), migraine with typical aura, migraine with prolonged aura, migraine without headache, hemiplegic migraine (e.g., familial hemiplegic migraine), basilar migraine (e.g., basilar artery migraine), carotidynia, abdominal migraine (e.g., periodic syndrome), hormonal migraine (e.g., pregnancy-induced migraine), exertion migraine, migraine with acute onset aura, ophthalmoplegic migraine, status migrainous, transformed migraine, retinal migraine, nocturnal migraine, childhood periodic syndromes that may be precursors to or associated with migraine, benign paroxysmal vertigo of childhood, alternating hemiplegia of childhood, and migrainous infarction.

By “musculoskeletal symptoms” is meant symptoms affecting muscles, bones, joints, tendons, ligaments, bursae, or nerves that may manifest as tenderness, pain, stiffness, weakness, fatigue, heaviness, cramps, myalgia, myopathy, loss of sensation, tremor, loss of function, or muscle destruction, deterioration, or atrophy seen on inspection, imaging, or by release of muscle enzymes in the blood.

By “pharmaceutically acceptable carrier” is meant a carrier that is physiologically acceptable to the treated subject while retaining the therapeutic properties of the compound (e.g., a statin or analogs thereof and vitamin D or analogs thereof) with which it is administered. One exemplary pharmaceutically acceptable carrier substance is physiological saline. Other physiologically acceptable carriers and their formulations are known to one skilled in the art.

By “pharmaceutically acceptable salt” is meant salts that are, within the scope of sound medical judgment, suitable for use in contact with the tissues of a subject without undue toxicity, irritation, or allergic response, and are commensurate with a reasonable benefit/risk ratio. Pharmaceutically acceptable salts are well known in the art. The salts can be prepared in situ during the final isolation and purification of the therapeutic compounds of the invention or separately by reacting the free base function with a suitable organic acid. Representative acid addition salts include, e.g., acetate, ascorbate, aspartate, benzoate, citrate, digluconate, fumarate, glucoheptonate, glycerophosphate, hemisulfate, heptonate, hexanoate, hydrobromide, hydrochloride, hydroiodide, lactate, malate, maleate, malonate, mesylate, oxalate, phosphate, succinate, sulfate, tartrate, thiocyanate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to, ammonium, tetramethylammonium, tetraethylammonium, methylamine, dimethylamine, trimethylamine, triethylamine, and ethylamine.

By “reducing the likelihood of” is meant reducing the severity, the frequency, and/or the duration of a migraine or symptoms thereof. Reducing the likelihood of migraines is synonymous with migraine prophylaxis or the chronic treatment of migraines. “Reducing the likelihood of” may also include reducing the severity, the frequency, or both the severity and frequency of a condition characterized by cardiovascular risk or endothelial dysfunction and musculoskeletal symptoms.

By “sequentially administering” is meant administering the therapeutic agents at separately staggered times (e.g., within 1 hour, 4 hours, 12 hours, 1 day, 7 days, or within 14 days of each other). Thus, the therapeutic agents (e.g., a statin and vitamin D) can be sequentially administered such that the beneficial pharmaceutical effect of the statin and vitamin D are realized by the subject at substantially the same time.

By “simultaneously administering” is meant administering the therapeutic agents substantially concurrently. The term “simultaneously administering” encompasses not only administering the two or more therapeutic agents (e.g., a statin and vitamin D) in a single pharmaceutical dosage form, but also the administration of each active agent in its own separate pharmaceutical dosage formulation. Where separate dosage formulations are used, the therapeutic agents can be administered at essentially the same time.

By “statin” is meant a compound capable of inhibiting the enzyme HMG-CoA reductase. Members of the statin family include naturally occurring and synthetic molecules, e.g., atorvastatin; cerivastatin; fluvastatin; lovastatin; rosuvastatin; acitemate; amlodipine/atorvastatin; BAY102987; BAY X 2678; BB476; bervastatin; BMY21950; BMY22089; colestolone; CP83101; dalvastatin; DMP565; simvastatin; ezetimibe/simvastatin; glenvastatin; L659699; L669262; mevastatin; nicotinic acid/lovastatin; nicotinic acid/simvastatin; P882222; P882284; PD134965; PD135022; pitavastatin; pravastatin; rosuvastatin; RP61969; S2468; SC37111; SC45355; simvastatin; SQ33600; SR12813; SR45023A; U20685; and U88156.

By “subject” is meant any animal, e.g., a mammal (e.g., a human). A subject who is being treated for, e.g., a migraine or a condition characterized by increased cardiovascular risk or endothelial dysfunction and musculoskeletal symptoms is one who has been diagnosed by a medical practitioner as having such a condition or being predisposed to such a condition. Subjects of the invention may also include those that do not presently have a migraine or a condition characterized by increased cardiovascular risk or endothelial dysfunction and musculoskeletal symptoms. Diagnosis may be performed by any suitable means, such as those described herein. One in the art will understand that subjects treated using the compositions or methods of the present invention may have been subjected to standard tests or may have been identified, without examination, as one at high risk due to the presence of one or more risk factors, such as age, genetics, or family history.

By “systemic administration” is meant any non-dermal route of administration and specifically excludes topical and transdermal routes of administration.

By “therapeutic agent” is meant any agent that produces a healing, curative, stabilizing, or ameliorative effect.

By “treating” or “ameliorating” is meant treating or ameliorating a condition or symptom(s) of the condition (e.g., migraine headache or a condition characterized by increased cardiovascular risk or endothelial dysfunction and musculoskeletal symptoms) before or after its onset. Symptoms of migraines include, e.g., severe headache, nausea, muscle tenderness, abdominal pain, visual aura, sensory hyperexcitability (e.g., photophobia, phonophobia, or osmophobia), blurred vision, nasal congestion, diarrhea, polyuria, pallor, sweating, localized edema of the scalp or face, scalp tenderness, prominence of a vein or artery in the temple, stiffness or tenderness of the neck, impairment of concentration or mood, vertigo, lightheadedness, fatigue, depression, and euphoria. Symptoms of a condition characterized by endothelial dysfunction and musculoskeletal symptoms (e.g., statin-associated musculoskeletal pain or weakness, arthritis, neuromuscular disease, a condition associated with stroke rehabilitation, or a condition associated with myocardial infarction rehabilitation) include, e.g., muscle tenderness, joint pain or inflammation, muscular weakness, loss of muscular control, myoclonus, myalgia, and myopathy. As compared with an equivalent untreated control, such amelioration or degree of treatment is at least 5%, 10%, 20%, 30%, 40%, 50%, 60%, 70%, 80%, 90%, 95%, 99%, or 100%, as measured by any standard technique.

By “vitamin D or analogs thereof” is meant a compound that binds to the vitamin D receptor (VDR). Vitamin D compounds and analogs thereof include, without limitation, vitamin D₁ (ergocalciferol with lumisterol); vitamin D₂ (ergocalciferol); vitamin D₃ (cholecalciferol); vitamin D₄ (22-dihydroergocalciferol); vitamin D₅ (sitocalciferol); doxercalciferol; calcitriol, α-calcidol; seocalcitol; calcipotriol; maxacalcitol; falecalcitriol; paricalcitol; alfacalcidol; calcifediol; dihydrotachysterol; ergosterol; 1α,24-dihydroxyvitamin D₄; 1α,24-dihydroxyvitamin D₂; 1α,25-dihydroxyvitamin D₂; 1α,25-dihydroxyvitamin D₄; or 1α,24,25-dihydroxyvitamin D₂.

Other features and advantages of the invention will be apparent from the following Detailed Description and the claims.

### DETAILED DESCRIPTION OF THE INVENTION

We describe compositions and methods for treating or reducing the likelihood of a migraine, reducing the frequency of migraines, reducing the duration of migraines, and ameliorating the symptoms of a migraine. The methods and compositions of the present invention may also be used to treat or prevent a condition characterized by increased cardiovascular risk or endothelial dysfunction and musculoskeletal symptoms (e.g., statin-associated musculoskeletal pain or weakness, arthritis, neuromuscular disease, a condition associated with stroke rehabilitation, or a condition associated with myocardial infarction rehabilitation).

### Therapeutic Agents

The co-administration of a statin or analogs thereof and vitamin D or analogs thereof may be used for the treatment of any subject that has been diagnosed with migraines, for the prevention of migraines, for reducing the frequency of migraines, or for the amelioration of symptoms (e.g., headaches) associated with migraines. The co-administration of a statin or analogs thereof and vitamin D or analogs thereof may also be used for the treatment of a condition characterized by increased cardiovascular risk or endothelial dysfunction and musculoskeletal symptoms (e.g., statin-associated musculoskeletal pain or weakness, arthritis, neuromuscular disease, a condition associated with stroke rehabilitation, or a condition associated with myocardial infarction rehabilitation).

### Statins and Analogs Thereof

Statins may be used as therapeutic agents in the compositions and methods of the invention. Statins of the invention include, e.g., atorvastatin; cerivastatin; fluvastatin; lovastatin; rosuvastatin; acitemate; amlodipine/atorvastatin; BAY102987; BAY X 2678; BB476; bervastatin; BMY21950; BMY22089; colestolone; CP83101; dalvastatin; DMP565; simvastatin; ezetimibe/simvastatin; glenvastatin; L659699; L669262; mevastatin; nicotinic acid/lovastatin; nicotinic acid/simvastatin; P882222; P882284; PD134965; PD135022; pitavastatin; pravastatin; rosuvastatin; RP61969; S2468; SC37111; SC45355; simvastatin; SQ33600; SR12813; SR45023A; U20685; and U88156.

Additional statins and analogs thereof are described, for example, in U.S. Pat. Nos. 3,983,140; 4,231,938; 4,282,155; 4,293,496; 4,294,926; 4,319,039; 4,343,814; 4,346,227; 4,351,844; 4,361,515; 4,376,863; 4,444,784; 4,448,784; 4,448,979; 4,450,171; 4,503,072; 4,517,373; 4,661,483; 4,668,699; 4,681,893; 4,719,229; 4,738,982; 4,739,073; 4,766,145; 4,782,084; 4,804,770; 4,841,074; 4,847,306; 4,857,546; 4,857,547; 4,940,727; 4,946,864; 5,001,148; 5,006,530; 5,075,311; 5,112,857; 5,116,870; 5,120,848; 5,166,364; 5,173,487; 5,177,080; 5,273,995; 5,276,021; 5,369,123; 5,385,932; 5,502,199; 5,763,414; 5,877,208; and 6,541,511, and U.S. Patent Application Publication Nos. 2002/0013334; 2002/0028826; 2002/0061901; and 2002/0094977, hereby incorporated by reference.

### Vitamin D and Analogs Thereof

Vitamin D and analogs thereof may be used as therapeutic agents in the compositions and methods of the invention. Vitamin D compounds and analogs thereof include, e.g., vitamin D₁ (ergocalciferol with lumisterol); vitamin D₂ (ergocalciferol); vitamin D₃ (cholecalciferol); vitamin D₄ (22-dihydroergocalciferol); vitamin D₅ (sitocalciferol); doxercalciferol; calcitriol, α-calcidol; seocalcitol; calcipotriol; maxacalcitol; falecalcitriol; paricalcitol; alfacalcidol; calcifediol; dihydrotachysterol; ergosterol; 1α,24-dihydroxyvitamin D₄; 1α,24-dihydroxyvitamin D₂; 1α,25-dihydroxyvitamin D₂; 1α,25-dihydroxyvitamin D₄; or 1α,24,25-dihydroxyvitamin D₂.

Additional vitamin D compounds and analogs thereof are described, for example, in U.S. Pat. Nos. 5,194,248; 6,103,709; 6,329,357; 6,706,725; 6,831,106; 7,205,420; 7,241,909; 7,259,143; 7,312,249; and 7,538,098.

### Additional Therapeutic Agents

If desired, the subject may also receive additional therapeutic regimens. For example, therapeutic agents may be administered with the therapeutic agents (e.g., statin and vitamin D or analog thereof) described herein at concentrations known to be effective for such therapeutic agents. Particularly useful agents include those that treat or ameliorate symptoms of a migraine. Exemplary agents are analgesics (e.g., non-steroidal anti-inflammatory drugs; acetaminophen; or opiates); β-blockers (e.g., alprenolol; bucindolol; carteolol; carvedilol; labetalol; nadolol; penbutolol; pindolol; propranolol; timolol; acebutolol; atenolol; betaxolol; bisoprolol; celiprolol; esmolol; metoprolol; nebivolol; and butaxamine); calcium channel blockers (e.g., mibefradil; bepridil; fluspirilene; diltiazem; verapamil; gallopamil; amlodipine; aranidipine; azelnidipine; barnidipine; benidipine; cilnidipine; clevidipine; efonidipine; felodipine; lacidipine; lercanidipine; manidipine; nicardipine; nifedipine; nilvadipine; nimodipine; nisoldipine; nitrendipine; and pranidipine); antiemetics (e.g., 5-HT₃ receptor antagonists (e.g., dolasetron and ondansetron); dopamine antagonists (e.g., metoclopramide); or antihistamines); serotonin receptor agonists (e.g., azapirones and triptans); barbiturates; antidepressants (e.g., monoamine oxidase inhibitors; tricyclic antidepressants (e.g., amitriptyline; butriptyline; clomipramine; desipramine; dosulepin; doxepin; imipramine; lofepramine; nortriptyline; protriptyline; and trimipramine); tetracyclic antidepressants; selective serotonin reuptake inhibitors; and serotonin-norepinephrine reuptake inhibitors); ergot alkaloids; steroids; anxiolytics (e.g., benzodiazepines (e.g., alprazolam; chlordiazepoxide; clonazepam; diazepam; or lorazepam); azapirones (e.g., buspirone and tandospirone); and hydroxyzine); amphetamines; nitric oxide synthase (NOS) inhibitors; narcotics; or anticonvulsants (e.g., carbamazepine; GABA analogs; and valproic acid).

The therapeutic agents of the invention may be admixed with additional active or inert ingredients, e.g., in conventional pharmaceutically acceptable carriers. A pharmaceutical carrier can be any compatible, non-toxic substance suitable for the administration of the compositions of the present invention to a subject (e.g., a human). Pharmaceutically acceptable carriers include, for example, water, saline, buffers, and other compounds described, for example, in the Merck Index, Merck & Co., Rahway, N.J.

In addition to the administration of therapeutic agents, additional therapeutic regimen may involve, e.g., a modification to the lifestyle of the subject being treated. Such lifestyle changes may be helpful to control migraines and include, e.g., increased exercise and avoidance of migraine triggers (e.g., food triggers or stress). Other lifestyle changes may include, e.g., weight loss, nutritional supplementation (e.g., glucosamine or chondroitin), the use of orthotics, and acupuncture.

### Formulation

The co-administration of a statin or analogs thereof and vitamin D or analogs thereof may be used for the treatment of any subject that has been diagnosed with migraines, for the prevention of migraines, for reducing the frequency of migraines, or for the amelioration of symptoms (e.g., headaches) associated with migraines. The co-administration of a statin or analogs thereof and vitamin D or analogs thereof may also be used for the treatment of any subject that has been diagnosed with a condition characterized by increased cardiovascular risk or endothelial dysfunction and musculoskeletal symptoms, for the prevention of such a condition, or for the amelioration of symptoms associated with such a condition.

Any of the therapeutic agents employed according to the present invention may be contained in any appropriate amount in any suitable carrier substance and is generally present in an amount of 1-95% by weight of the total weight of the composition. Therapeutic formulations are prepared using standard methods known in the art by mixing the active ingredient having the desired degree of purity with physiologically acceptable carriers, excipients, or stabilizers in the form of lyophilized formulations or aqueous solutions (see, e.g., Remington's Pharmaceutical Sciences, 20^{th} edition, Ed. A. Gennaro, 2000, Lippincott, Williams & Wilkins, Philadelphia, Pa.). Acceptable carriers include, e.g., saline; buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagines, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

The formulations of the present invention may contain a pharmaceutically acceptable salt (e.g., sodium chloride) at physiological concentrations. The formulations of the invention can contain a pharmaceutically acceptable preservative. In some embodiments, the preservative concentration ranges from 0.1 to 2.0% v/v. Suitable preservatives include those known in the pharmaceutical arts. Benzyl alcohol, phenol, m-cresol, methylparaben, and propylparaben are preferred preservatives. The formulations of the invention can include a pharmaceutically acceptable surfactant. Surfactants may include, e.g., non-ionic detergents, Tween-20, and pluronic acid (F68). Suitable surfactant concentrations are, e.g., 0.005 to 0.02%.

The composition may be provided in a dosage form that is, e.g., suitable for the oral (e.g., sublingual), parenteral (e.g., intravenous or intramuscular), rectal, cutaneous, nasal, vaginal, inhalant, skin (e.g., via a skin patch), or ocular administration route. Thus, the composition may be in the form of, e.g., tablets, capsules, pills, powders, granulates, suspensions, emulsions, solutions, gels (e.g., hydrogels), pastes, ointments, creams, plasters, drenches, osmotic delivery devices, suppositories, enemas, injectables, implants, sprays, or aerosols

For intranasal administration or administration by inhalation, the compounds of the invention may be delivered in the form of, e.g., a solution or suspension from a pump spray container that is squeezed or pumped by the patient or as an aerosol spray from a pressurized container or a nebulizer with the use of a suitable propellant, e.g., dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base, such as lactose or starch.

Where sustained-release administration is desired, microencapsulation of the therapeutic agents of the present invention is contemplated. The sustained-release formulations may include those developed using poly-lactic-coglycolic acid (PLGA) polymer. The degradation products of PLGA, lactic and glycolic acid, can be cleared quickly from the human body. Moreover, the degradability of this polymer can be adjusted from months to years depending on its molecular weight and composition.

Each agent of the present invention may be formulated in a variety of ways that are known in the art. Desirably, the therapeutic agents are formulated together for the simultaneous or near simultaneous administration of the therapeutic agents. Such co-formulated compositions can include the two agents formulated together in the same, e.g., pill, capsule, or liquid. It is to be understood that, when referring to the formulation of such combinations, the formulation technology employed is also useful for the formulation of the individual agents of the combination, as well as other combinations of the invention. By using different formulation strategies for different agents, the pharmacokinetic profiles for each agent can be suitably matched.

The individually or separately formulated agents can be packaged together as a kit. Non-limiting examples include kits that contain, e.g., two pills, a pill and a powder, a suppository and a liquid in a vial, or two topical creams. The kit can include optional components that aid in the administration of the unit dose to patients, such as vials for reconstituting powder forms, syringes for injection, customized intravenous delivery systems, or inhalers. Additionally, the unit dose kit can contain instructions for the preparation and administration of the compositions. The kit may be, e.g., manufactured as a single use unit dose for one subject, multiple uses for a particular subject (e.g., at a constant dose or in which the individual compounds may vary in potency as therapy progresses), or the kit may contain multiple doses suitable for administration to multiple subjects (e.g., bulk packaging). The kit components may be assembled in, e.g., cartons, blister packs, bottles, or tubes.

### Dosages

Generally, when administered to a human, the dosage of any of the therapeutic agents of the invention will depend on the nature of the agent and can readily be determined by one skilled in the art. Typically, such dosage is normally about 0.001 mg to 2000 mg per day, desirably about 1 mg to 1000 mg per day, and more desirably about 5 mg to 500 mg per day.

Administration of each drug, alone or in combination, can be one to four times daily for one day to one year and may even be for the life of the subject. Chronic, long-term administration will be required in some cases.

Appropriate dosages of compounds used in the methods described herein depend on several factors, including the administration method, the severity of the disorder (e.g., migraine), and the age, weight, and health of the subject to be treated. Additionally, pharmacogenomic information (e.g., the effect of genotype on the pharmacokinetic, pharmacodynamic, or efficacy profile of a therapeutic) about a particular subject may affect the dosage used.

### OTHER EMBODIMENTS

From the foregoing description, it will be apparent that variations and modifications may be made to the invention described herein to adopt it to various usages and conditions. Such embodiments are also within the scope of the following claims.

All publications, patent applications, and patents mentioned in this specification are herein incorporated by reference to the same extent as if each independent publication, patent application, or patent was specifically and individually indicated to be incorporated by reference.

From the foregoing description, one skilled in the art can easily ascertain the essential characteristics of this invention; can make various changes and modifications of the invention to adapt it to various usages and conditions. Thus, other embodiments are also within the claims.

What is claimed is:

1. A method of treating or reducing the likelihood of a migraine in a subject in need thereof, said method comprising administering to said subject (i) a statin or analogs thereof and (ii) vitamin D or analogs thereof, wherein said statin or analogs thereof and said vitamin D or analogs thereof are administered in amounts and for a duration that together are sufficient to treat or reduce the likelihood of a migraine in said subject.

2. A method of ameliorating the symptoms of a migraine in a subject in need thereof, said method comprising administering to said subject (i) a statin or analogs thereof and (ii) vitamin D or analogs thereof, wherein said statin or analogs thereof and said vitamin D or analogs thereof are administered in amounts and for a duration that together are sufficient to ameliorate the symptoms of a migraine in said subject.

3. A method of reducing the frequency or duration of migraines in a subject in need thereof, said method comprising administering to said subject (i) a statin or analogs thereof and (ii) vitamin D or analogs thereof, wherein said statin or analogs thereof and said vitamin D or analogs thereof are administered in amounts and for a duration that together are sufficient to reduce the frequency or duration of migraines in said subject.

4. A method of treating a condition characterized by increased cardiovascular risk or endothelial dysfunction and musculoskeletal symptoms in a subject in need thereof, said method comprising administering to said subject (i) a statin or analogs thereof and (ii) vitamin D or analogs thereof, wherein said statin or analogs thereof and said vitamin D or analogs thereof are administered in amounts and for a duration that together are sufficient to treat said condition.

5. The method of claim 4, wherein said condition is statin-associated musculoskeletal pain or weakness, arthritis, neuromuscular disease, a condition associated with stroke rehabilitation, or a condition associated with myocardial infarction rehabilitation.

6. The method of any one of claims 1-5, wherein said statin is atorvastatin; cerivastatin; fluvastatin; lovastatin; rosuvastatin; acitemate; amlodipine/atorvastatin; BAY102987; BAY X 2678; BB476; bervastatin; BMY21950; BMY22089; colestolone; CP83101; dalvastatin; DMP565; ezetimibe/simvastatin; glenvastatin; L659699; L669262; mevastatin; nicotinic acid/lovastatin; nicotinic acid/simvastatin; P882222; P882284; PD134965; PD135022; pitavastatin; rosuvastatin; RP61969; S2468; SC37111; SC45355; simvastatin; SQ33600; SR12813; SR45023A; U20685; and U88156.

7. The method of claim 6, wherein said statin is simvastatin.

8. The method of any one of claims 1-7, wherein said vitamin D or vitamin D analog is doxercalciferol, calcitriol, α-calcidol, seocalcitol, calcipotriol, maxacalcitol, falecalcitriol, paricalcitol, alfacalcidol, calcifediol, cholecalciferol, dihydrotachysterol, ergosterol, ergocalciferol, 1α,2-dihydroxyvitamin D₄, 1α,24-dihydroxyvitamin D₂, 1α,25-dihydroxyvitamin D₂, 1α,25-dihydroxyvitamin D₄, or 1α,24,25-dihydroxyvitamin D₂.

9. The method of claim 8, wherein said vitamin D or vitamin D analog is cholecalciferol or ergocalciferol.

10. The method of claim 9, wherein said vitamin D or vitamin D analog is cholecalciferol.

11. The method of claim 9, wherein said vitamin D or vitamin D analog is ergocalciferol.

12. The method of any of claims 9-11, wherein said statin is simvastsatin.

13. The method of any one of claims 1-12, wherein said statin or analogs thereof and said vitamin D or analogs thereof are administered simultaneously or sequentially.

14. The method of any one of claims 1-3, wherein said method further comprises administering an additional therapeutic agent to said subject.

15. The method of claim 14, wherein said additional therapeutic agent is an analgesic, a β-blocker, a calcium channel blocker, an antiemetic, a serotonin receptor agonist, a barbiturate, an antidepressant, an ergot alkaloid, a steroid, an anxiolytic, an amphetamine, a NOS inhibitor, a narcotic, or an anticonvulsant.

16. The method of claim 15, wherein said analgesic is a non-steroidal anti-inflammatory agent.

17. The method of claim 15, wherein said antidepressant is a tricyclic antidepressant.

18. The method of claim 15, wherein said anticonvulsant is valproic acid.

19. The method of any one of claims 1-18, wherein said subject is a human subject.

20. A pharmaceutical composition comprising (i) a statin or analogs thereof, (ii) vitamin D or analogs thereof, and (iii) a pharmaceutically acceptable carrier, wherein said statin or analogs thereof and said vitamin D or analogs thereof are present in an amount that, when administered to a subject, are sufficient to treat or reduce the likelihood of a migraine in said subject.

21. A pharmaceutical composition comprising (i) a statin or analogs thereof, (ii) vitamin D or analogs thereof, and (iii) a pharmaceutically acceptable carrier, wherein said statin or analogs thereof and said vitamin D or analogs thereof are present in an amount that, when administered to a subject, are sufficient to ameliorate the symptoms of a migraine in said subject.

22. A pharmaceutical composition comprising (i) a statin or analogs thereof, (ii) vitamin D or analogs thereof, and (iii) a pharmaceutically acceptable carrier, wherein said statin or analogs thereof and said vitamin D or analogs thereof are present in an amount that, when administered to a subject, are sufficient to reduce the frequency or duration of migraines in said subject.

23. A pharmaceutical composition comprising (i) a statin or analogs thereof, (ii) vitamin D or analogs thereof, and (iii) a pharmaceutically acceptable carrier, wherein said statin or analogs thereof and said vitamin D or analogs thereof are present in an amount that, when administered to a subject, are sufficient to treat a condition characterized by increased cardiovascular risk or endothelial dysfunction and musculoskeletal symptoms in said subject.

24. The pharmaceutical composition of claim 23, wherein said condition is statin-associated musculoskeletal pain or weakness, arthritis, neuromuscular disease, stroke rehabilitation, or myocardial infarction rehabilitation.

25. The pharmaceutical composition of any one of claims 20-24, wherein said statin or analogs thereof and said vitamin D or analogs thereof are formulated in a single composition.

26. The pharmaceutical composition of any one of claims 20-25, wherein said statin or analogs thereof and said vitamin D or analogs thereof are formulated for oral administration.

27. The pharmaceutical composition of any one of claims 20-25, wherein said statin or analogs thereof and said vitamin D or analogs thereof are formulated for systemic administration.

28. The pharmaceutical composition of any one of claims 20-25, wherein said statin or analogs thereof and said vitamin D or analogs thereof are formulated for inhalation.

29. The pharmaceutical composition of any one of claims 20-25, wherein said statin or analogs thereof and said vitamin D or analogs thereof are formulated for topical application.

30. The pharmaceutical composition of any one of claims 20-29, wherein said statin is atorvastatin; cerivastatin; fluvastatin; lovastatin; rosuvastatin; acitemate; amlodipine/atorvastatin; BAY102987; BAY X 2678; BB476; bervastatin; BMY21950; BMY22089; colestolone; CP83101; dalvastatin; DMP565; ezetimibe/simvastatin; glenvastatin; L659699; L669262; mevastatin; nicotinic acid/lovastatin; nicotinic acid/simvastatin; P882222; P882284; PD134965; PD135022; pitavastatin; rosuvastatin; RP61969; S2468; SC37111; SC45355; simvastatin; SQ33600; SR12813; SR45023A; U20685; and U88156.

31. The pharmaceutical composition of claim 30, wherein said statin is simvastatin.

32. The pharmaceutical composition of any one of claims 20-31, wherein said vitamin D or vitamin D analog is doxercalciferol, calcitriol, α-calcidol, seocalcitol, calcipotriol, maxacalcitol, falecalcitriol, paricalcitol, alfacalcidol, calcifediol, cholecalciferol, dihydrotachysterol, ergosterol, ergocalciferol, 1α,2-dihydroxyvitamin D₄, 1α,24-dihydroxyvitamin D₂, 1α,25-dihydroxyvitamin D₂, 1α,25-dihydroxyvitamin D₄, or 1α,24,25-dihydroxyvitamin D₂.

33. The pharmaceutical composition of claim 32, wherein said vitamin D or vitamin D analog is cholecalciferol or ergocalciferol.

34. The pharmaceutical composition of claim 33, wherein said vitamin D or vitamin D analog is cholecalciferol.

35. The pharmaceutical composition of claim 33, wherein said vitamin D or vitamin D analog is ergocalciferol.

36. The method of any of claims 33-35, wherein said statin is simvastatin.

37. The pharmaceutical composition of any one of claims 20-36, wherein said composition further comprises an additional therapeutic agent.

38. The pharmaceutical composition of claim 37, wherein said additional therapeutic agent is an analgesic, a β-blocker, a calcium channel blocker, an antiemetic, a serotonin receptor agonist, a barbiturate, an antidepressant, an ergot alkaloid, a steroid, an anxiolytic, an amphetamine, a NOS inhibitor, a narcotic, or an anticonvulsant.

39. The pharmaceutical composition of claim 38, wherein said analgesic is a non-steroidal anti-inflammatory agent.

40. The pharmaceutical composition of claim 38, wherein said antidepressant is a tricyclic antidepressant.

41. The pharmaceutical composition of claim 38, wherein said anticonvulsant is valproic acid.
